(19) United States
(12) Patent Application Publication
(10) Pub. No.: US 2024/0099659 A1
(43) Pub. Date: Mar. 28, 2024

(54) Automated Measurement System

(71) Applicant: Bard Access Systems, Inc., Salt Lake City, UT (US)

(72) Inventors: Steffan Sowards, Salt Lake City, UT (US); William Robert McLaughlin, Bountiful, UT (US)

(21) Appl. No.: 18/371,629

(22) Filed: Sep. 22, 2023

Related U.S. Application Data
(60) Provisional application No. 63/409,544, filed on Sep. 23, 2022.

(51) Int. Cl. A61B 5/00

(52) U.S. Cl. CPC A61B 5/6813; A61B 2560/06

(57) ABSTRACT

Embodiments described herein are directed to an automatic catheter measurement system for determining a length of a catheter required to extend between an insertion site and a target location, prior to placement of the catheter. The system can include a measurement device that can be aligned with, and map, a three-dimensional arrangement of one or more external landmarks. The measurement device can include magnetic and/or fiber optic systems to map the external landmarks. The system then determines a framework to provide a predicted catheter length required to extend between the insertion site and target location. The measurement device can also be included with the catheter during placement to confirm the actual catheter length and improve the accuracy of future predicted frameworks.

### PRIORITY

This application claims the benefit of priority to U.S. Provisional Application No. 63/409,544, filed Sep. 23, 2022, which is incorporated by reference in its entirety into this application.

### SUMMARY

Briefly summarized, embodiments disclosed herein are directed to an automated measurement tool using digital landmarking for automated measuring of intravascular pathways.

When placing central venous catheters (CVC), peripherally inserted central catheters (PICC), or the like, the position of the distal tip of the catheter relative to a target location can be crucial to the efficacy of the treatment. For example, where a target location is a lower ⅓^{rd} of the Superior Vena Cava (“SVC”), if the distal tip is placed proximally of the target location the efficacy of the medication can be reduced. If the distal tip is placed distally of the target location the distal tip can cause arrhythmia.

For each placement procedure, the length of the catheter required to place the distal tip at the target location can vary. This is because the distances between the target location, the insertion site into the vasculature, and the placement of the access point (i.e. insertion site into the body of the patient) can vary between different patient anatomies and individual placement procedures. As such, the length of the catheter must be estimated prior to placement and trimmed to a suitable length.

Current methods for estimating catheter length include using a measuring tape to measure line-of-sight distances between external physical landmarks and estimating a length of the intravascular pathway based on these separate measurements. Exemplary external landmarks can include the insertion site (access point), the shoulder, the clavicular head, and the third intercostal space. As will be appreciated, such methods are subject to various estimation errors leading to misplacement of the distal tip of the catheter.

Embodiments disclosed herein include a system having an elongate measurement device (“device”) that can be placed externally on the patient and aligned with one or more external physical landmarks such as the insertion site, the shoulder, the clavicular head, and the third intercostal space. The measurement device can then determine the three-dimensional spatial relationship and distances between these external landmarks using, for example, Bragg grated fiber optics, and/or magnetic signature tracking, to create a digital framework prior to catheter placement.

Using the digital framework, the system can then predict the three-dimensional shape of the intravascular pathway between the insertion site and the target location and determine an anticipated length of the catheter required to place the distal tip at the target location. Further, the measurement device can be included with the catheter during the placement procedure to track the actual three-dimensional shape of the intravascular pathway during the placement procedure. The system can then compare the predicted pathway with the actual pathway to update the digital framework and to increase the accuracy of future digital frameworks.

Disclosed herein is a system for determining a catheter length required to extend between an insertion site and a target location prior to placement within a patient body, the system including, a measurement device including an optical fiber having one or more of core fibers and configured to be aligned with one or more external landmarks, and a console including one or more processors and a non-transitory computer-readable medium having stored thereon logic, when executed by the one or more processors, causes operations including, providing an incident light signal to the optical fiber, receiving a reflected light signal of the incident light, processing the reflected light signal to determine a location of one or more external landmarks in three-dimensional space, and determining a predicted catheter length required to extend intravascularly between the insertion site and the target location.

In some embodiments, the measurement device includes a stylet having a multi-core optical fiber extending therethrough and one or both of a magnetic element and an electrically conductive element extending therethrough.

In some embodiments, an external landmark of the one or more external landmarks includes an insertion site, a shoulder, a clavicle head, and a third intercostal space.

In some embodiments, the console is further configured to receive an input to indicate when a portion of the measurement device is aligned an external landmark of the one or more external landmarks.

In some embodiments, the measurement device further includes a pressure sensor array extending along a longitudinal length thereof, and wherein activation of a sensor of the pressure sensor array indicates that a portion of the measurement device is aligned with an external landmark of the one or more external landmarks.

In some embodiments, the console is further configured to process the reflected light signal to determine a location, angle, and direction of one or more inflections in the measurement device and determine a location of an external landmark of the one or more external landmarks in three-dimensional space.

In some embodiments, the measurement device displays malleable physical qualities and can be shaped into a shape and remain in that shape until reshaped.

In some embodiments, determining the predicted catheter length further includes determining one or more of a distance, an angle, and a direction between a first external landmark and a second external landmark.

Also disclosed is a method of determining a catheter length required to extend between an insertion site and an intravascular target location prior to placement within a patient body including, aligning a measurement device with a first external landmark and a second external landmark on a surface of the patient body, the measurement device including one or both of an optical fiber having one or more of core fibers and a magnetic signature region, mapping a three dimensional location of the first external landmark relative to the second external landmark, and determining a predicted length (L_{P}) of an intravascular pathway between the insertion site and the intravascular target location that extends proximate the first landmark and the second landmark.

In some embodiments, the method further includes extending the measurement device intravascularly from the insertion site to the target location and mapping an intravascular pathway therebetween to determine an actual length (L_{A}) of the intravascular pathway between the insertion site and the target location.

In some embodiments, the measurement device is configured to be placed within a lumen of a catheter.

In some embodiments, the measurement device includes a stylet having a multi-core optical fiber extending therethrough and one or both of a magnetic element and an electrically conductive element extending therethrough.

In some embodiments, the first external landmark or the second external landmark includes one of an insertion site, a shoulder, a clavicle head, and a third intercostal space.

In some embodiments, aligning a measurement device further includes receiving an input to indicate when a portion of the measurement device is aligned one of the first external landmark or the second external landmark.

In some embodiments, the measurement device further includes a pressure sensor array extending along a longitudinal length thereof, and wherein the method further includes activation of a sensor of the pressure sensor array to indicate that a portion of the measurement device is aligned with one of the first external landmark or the second external landmark.

In some embodiments, mapping a three dimensional location of a first external landmark or a second external landmark further includes processing a reflected light signal to determine a location, angle, and direction of an inflection in the measurement device.

In some embodiments, aligning a measurement device further includes shaping the measurement device into a shape, the measurement device remaining in that shape until reshaped.

In some embodiments, mapping a three dimensional location of a first external landmark or a second external landmark further includes identifying a first magnetic signature region and a second magnetic signature region and tracking the first magnetic signature region and a second magnetic signature region in three-dimensional space.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows an exemplary automated measurement tool system, in accordance with embodiments disclosed herein.

FIG. 2A shows an exemplary measurement device for use with the system of FIG. 1, in accordance with embodiments disclosed herein.

FIG. 2B shows an exemplary measurement device and catheter assembly for use with the system of FIG. 1, in accordance with embodiments disclosed herein.

FIG. 3A shows an exemplary measurement device aligned with exemplary physical landmarks, in accordance with embodiments disclosed herein.

FIG. 3B shows an exemplary measurement device including a sensor array aligned with exemplary physical landmarks, in accordance with embodiments disclosed herein.

FIG. 3C shows an exemplary measurement device aligned with exemplary physical landmarks, in accordance with embodiments disclosed herein.

FIG. 3D shows an exemplary digital framework mapping a three-dimensional arrangement of exemplary landmarks, in accordance with embodiments disclosed herein.

FIG. 4 shows an exemplary catheter placement procedure, in accordance with embodiments disclosed herein.

FIG. 5 shows a cross-sectional view of an exemplary measurement device, in accordance with embodiments disclosed herein.

FIG. 6A shows a perspective cut-away view of an exemplary measurement device, in accordance with embodiments disclosed herein.

FIG. 6B shows a cross-section view of the measurement device of FIG. 6A, in accordance with embodiments disclosed herein.

FIG. 7 shows an exemplary measurement device including a magnetic signature and aligned with exemplary physical landmarks, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

### Terminology

Regarding terms used herein, it should be understood the terms are for the purpose of describing some particular embodiments, and the terms do not limit the scope of the concepts provided herein. Ordinal numbers (e.g., first, second, third, etc.) are generally used to distinguish or identify different components or operations, and do not supply a serial or numerical limitation. For example, “first,” “second,” and “third” components or operations need not necessarily appear in that order, and the particular embodiments including such components or operations need not necessarily be limited to the three components or operations. Similarly, labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The term “logic” is representative of hardware and/or software that is configured to perform one or more functions. As hardware, logic may include circuitry having data processing and/or storage functionality. Examples of such circuitry may include, but are not limited or restricted to a processor, a programmable gate array, a microcontroller, an application specific integrated circuit, combinatorial circuitry, or the like. Alternatively, or in combination with the hardware circuitry described above, the logic may be software in the form of one or more software modules, which may be configured to operate as its counterpart circuitry. The software modules may include, for example, an executable application, a daemon application, an application programming interface (API), a subroutine, a function, a procedure, a routine, source code, or even one or more instructions. The software module(s) may be stored in any type of a suitable non-transitory storage medium, such as a programmable circuit, a semiconductor memory, non-persistent storage such as volatile memory (e.g., any type of random access memory “RAM”), persistent storage such as non-volatile memory (e.g., read-only memory “ROM”, power-backed RAM, flash memory, phase-change memory, etc.), a solid-state drive, hard disk drive, an optical disc drive, or a portable memory device.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

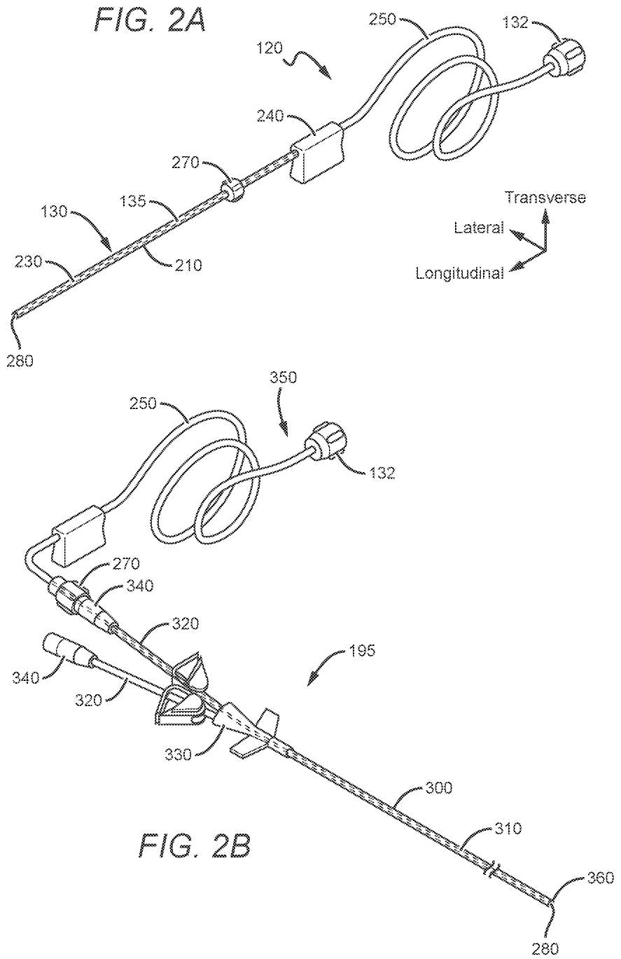

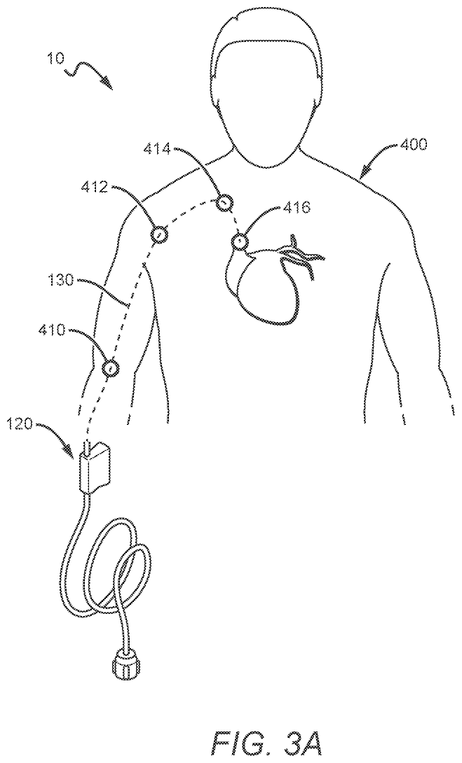

To assist in the description of embodiments described herein, as shown in FIG. 2A, a longitudinal axis extends substantially parallel to an axial length of the measurement device. A lateral axis extends normal to the longitudinal axis, and a transverse axis extends normal to both the longitudinal and lateral axes. As used herein a location of an inflection relates to a longitudinal position of the inflection along a longitudinal axis. An angle of an inflection is the degree of deflection from the longitudinal axis. A direction of an inflection is a radial direction of the inflection from a central longitudinal axis.

Unless defined otherwise, all technical and scientific terms used herein have the same meaning as commonly understood by those of ordinary skill in the art.

### Automated Measurement Systems

Automated catheter measurement systems can include a measurement device that includes fiber-optically enable shape sensing systems and/or magnetic signature tracking systems configured to map a three-dimensional shape of the elongate measurement device. The measurement device can be aligned with one or more external landmarks and the system can determine a three-dimensional shape (physical state) of the measurement device and thereby map a three-dimensional arrangement of the one or more landmarks. In an embodiment, the system can use a fiber-optic shape sensing system to determine a physical state of measurement device based on the number, location, angle, and direction of inflections along a longitudinal length thereof. The inflections can be detected by changes in the incidence of light that are reflected through fiber optic cables. Based on theses changes in light incidence, the system can determine a three-dimensional arrangement of the measurement device when aligned with one or more external landmarks.

In an embodiment, the system can employ a magnetic signature tracking system configured to recognize one or more portions of the measurement device based on a specific pattern magnetic dipoles. The system can be configured to identify and track these individual portions of the measurement device in three-dimensional space. When the one or more portions of the measurement device are aligned with the one or more external landmarks the system can map a three-dimensional arrangement of the one or more landmarks.

Once the external landmarks have been mapped in three-dimensional space, the system can use these reference points to determine a digital framework to estimate a length of an intravascular pathway between an insertion site and a target location. From this framework the system can determine a predicted catheter length required for the placement procedure.

In one embodiment, the system can use reflective gratings such as fiber Bragg gratings (“FBG”) distributed along a core fiber disposed in/on a stylet portion of the measurement device. An outgoing optical signal produced by a light source is incident on each of the FBGs along the core fiber, where each grating reflects light of a prescribed spectral width to produce a return optical signal to the console. According to one embodiment of the disclosure, shifts in wavelength of reflected light signals returned by each of the core fibers may be aggregated based on FBGs associated with the same cross-sectional region of the stylet (or specific spectral width) and a processor of the console may execute shape sensing analytic logic to perform analytics associated with the wavelength shifts (e.g., analysis of degree, comparison between wavelength shifts between periphery core fibers and the center core fiber or between periphery core fibers, etc.) to identify the physical state (three-dimensional shape) of the stylet. The data is communicated to a user of the console to identify (and render) its position, two-dimensional (2-D) shape, and/or three-dimensional (3-D) shape of the stylet along its length, form and shape (e.g., bending, torsion) as well as orientation. Such information can be presented by the console to the user. Further details regarding these and other embodiments are given hereafter.

In light of the above, a multi-core optical fiber can be paired with a conductive and/or magnetic medium within the stylet portion of the measuring device, to employ multiple modalities. For example, a first modality constitutes an optical modality with shape sensing functionality to determine the three-dimensional shape of the measurement device. Further, the measurement device can also employ additional modalities to also track the measurement device and/or map a physical state. These additional modalities can include magnetic signature tracking, a tip location/navigation system (“TLS”) modality, and an ECG modality.

The magnetic signature tracking system can be configured to triangulate a location of a portion of the measurement device (or stylet portion thereof) based on the relative strength of a magnetic field. The measurement device can include regions of magnetic dipoles that form a distinctive pattern, or “magnetic signature.” The system can determine one or more distinct magnetic signatures to differentiate between one or more portions of the measurement device. The system can then track these individual magnetic signatures in three dimensional space to provide a three-dimensional shape of the measurement device. A tip location/navigation system (“TLS”) modality can use a conductive medium configured to detect and avoid any tip malposition during placement. Lastly, an ECG modality can employ an ECG signal-based catheter tip guidance to enable tracking and guidance of the measurement device.

### Automatic Catheter Measurement Tool

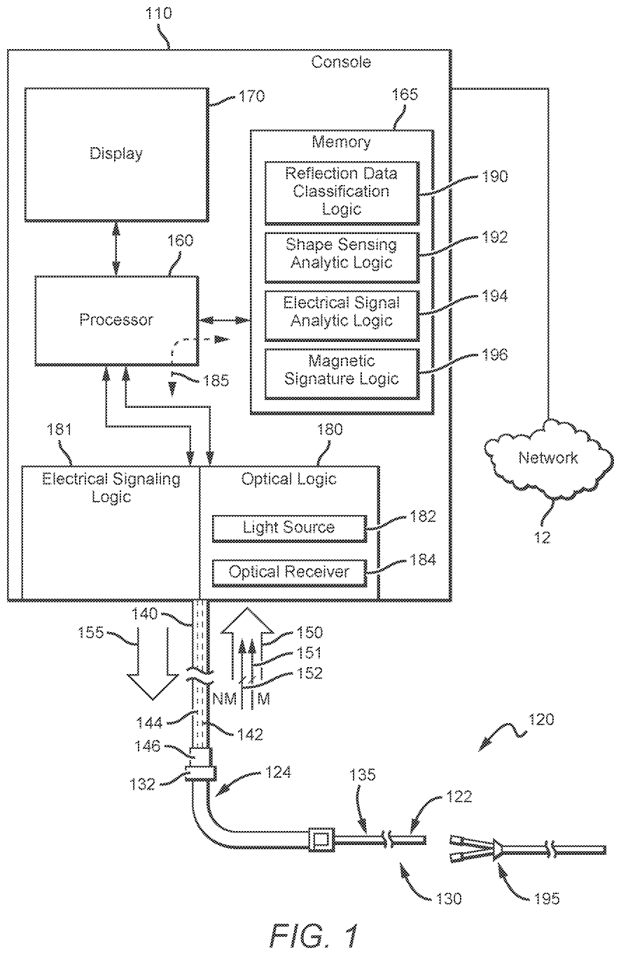

Referring to FIG. 1, an illustrative embodiment of an automatic catheter measurement system (“system”) 100 is shown. As shown, the system 100 generally includes a console 110 and an elongate measurement device (“device”) 120 communicatively coupled to the console 110.

For this embodiment, the measurement device 120 includes an elongate probe (e.g., stylet) 130 on its distal end 122 and a console connector 132 on its proximal end 124. The console connector 132 enables the measurement device 120 to be operably connected to the console 110 via an interconnect 140 including one or more optical fibers 142 (hereinafter, “optical fiber(s)”) and a conductive medium 144 terminated by a single optical/electric connector 146 (or terminated by dual connectors). Herein, the connector 146 is configured to engage (mate) with the console connector 132 to allow for the propagation of light between the console 110 and the measurement device 120 as well as the propagation of electrical signals from the stylet 130 to the console 110.

An exemplary implementation of the console 110 includes a processor 160, a memory 165, a display 170, and one or more logic engines such as an optical logic 180, an electrical signaling logic 181, a reflection data classification logic 190, a shape sensing analytic logic 192, an electrical signal analytic logic 194 and a magnetic signature logic 196. It will be appreciated, however, that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The processor 160, with access to the memory 165 (e.g., non-volatile memory), is included to control functionality of the console 110 during operation. As shown, the display 165 may be a liquid crystal diode (LCD) display integrated into the console 110 and employed as a user interface to display information to the clinician. In another embodiment, the display 165 may be separate from the console 110. Although not shown, a user interface is configured to provide user control of the console 110. In an embodiment, the content depicted by the display 165 may change according to which modality is being employed by the system 100: fiber-optical, magnetic signature, TLS, ECG, or other modality, or combination thereof.

In TLS mode, the content rendered by the display 165 may constitute a two-dimensional (2-D) or three-dimensional (3-D) representation of the physical state (e.g. shape, form, and/or orientation) of the measurement device 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below. In one embodiment, information from multiple modes, such as optical, magnetic, TLS or ECG for example, may be displayed concurrently (e.g., at least partially overlapping in time). In one embodiment, the display 165 is a liquid crystal diode (LCD) device.

Referring still to FIG. 1, the optical logic 180 is configured to support operability of the measurement device 120 and enable the return of information to the console 110, which may be used to determine the physical state associated with the stylet 130 along with monitored electrical signals such as ECG signaling via an electrical signaling logic 181 that supports receipt and processing of the received electrical signals from the measurement device 120 (e.g., ports, analog-to-digital conversion logic, etc.). The physical state of the measurement device 120 may be based on changes in characteristics of the reflected light signals 150 received from the measurement device 120. The characteristics may include shifts in wavelength caused by strain on certain regions of the core fibers integrated within a multi-core optical fiber 135 positioned within or operating as the probe 130, as shown below. From information associated with the reflected light signals 150, the console 110 may determine (through computation or extrapolation of the wavelength shifts) the physical state of the measurement device 120.

According to one embodiment of the disclosure, as shown in FIG. 1, the optical logic 180 may include a light source 182 and an optical receiver 184. The light source 182 is configured to transmit the broadband incident light 155 for propagation over the optical fiber(s) 142 included in the interconnect 140, which are optically connected to the multi-core optical fiber 135 within the measurement device 120. In one embodiment, the light source 182 is a tunable swept laser, although other suitable light source can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the multi-core optical fiber 135 deployed within the stylet 130 (see FIG. 2A), and (ii) translate the reflected light signals 150 into reflection data 185, namely data in the form of electrical signals representative of the reflected light signals including wavelength shifts caused by strain. The reflected light signals 150 associated with different spectral widths may include reflected light signals 151 provided from sensors positioned in the center core fiber (reference) of the multi-core optical fiber 135 and reflected light signals 152 provided from sensors positioned in the periphery core fibers of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data 185 to the memory 165 for storage and processing by reflection data classification logic 190. The reflection data classification logic 190 may be configured to: (i) identify which core fibers pertain to which of the received reflection data 185 and (ii) segregate the reflection data 185 provided from reflected light signals 150 pertaining to similar regions of the stylet 130 or spectral widths into analysis groups. The reflection data for each analysis group is made available to shape sensing analytic logic 192 for analytics.

According to one embodiment of the disclosure, the shape sensing analytic logic 192 is configured to compare wavelength shifts measured by sensors deployed in each periphery core fiber at the same measurement region of the stylet 130 (or same spectral width) to the wavelength shift at a center core fiber of the multi-core optical fiber 135 positioned along central axis and operating as a neutral axis of bending. From these analytics, the shape sensing analytic logic 192 may determine the shape the core fibers have taken in 3-D space and may further determine the current physical state of the measurement device 120 in 3-D space for rendering on the display 170.

According to one embodiment of the disclosure, the shape sensing analytic logic 192 may generate a rendering of the current physical state of the stylet 130, based on heuristics or run-time analytics. For example, the shape sensing analytic logic 192 may be configured in accordance with machine-learning techniques to access a data store (library) with pre-stored data (e.g., images, etc.) pertaining to different regions of the stylet 130 in which reflected light from core fibers have previously experienced similar or identical wavelength shifts. From the pre-stored data, the current physical state of the stylet 130 may be rendered. Alternatively, as another example, the shape sensing analytic logic 192 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the stylet 130.

The console 110 may further include electrical signal receiver logic 186, which is positioned to receive one or more electrical signals from the stylet 130. The stylet 130 is configured to support both optical connectivity as well as magnetic, electro-magnetic, and/or electrical connectivity.

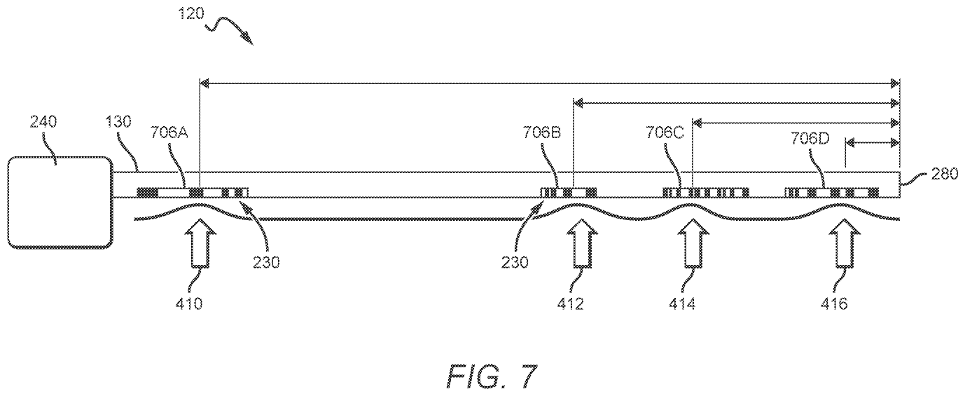

In an embodiment, the magnetic signature logic 196 can be configured to detect and analyze distinct pattern(s) of magnetic dipoles. Individual patterns of magnetic dipoles can be associated with individual regions of the measurement device 120 (FIG. 7). These individual magnetic signatures can be stored on magnetic signature logic 196. Once the magnetic signature logic 196 has identified the individual dipole region, the magnetic signature logic 196 can triangulate and track the region in three-dimensional space.

Further details and embodiments of magnetic signature tracking systems can be found in U.S. Provisional Application 63/250,022 filed Sep. 29, 2021, and U.S. Provisional Application 63/250,057 filed Sep. 29, 2021, each of which are incorporated by reference in their entirety, herein.

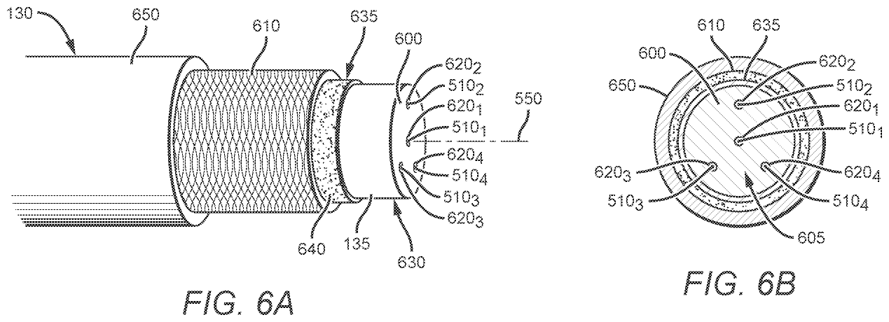

FIGS. 2A-2B show an exemplary embodiment of the elongate measurement device 120. In an embodiment, the measurement device 120 can generally include a probe such as a stylet 130 that includes, or is formed of, a multi-core optical fiber 135. Optionally, the measurement device 120 can be operably connected to a catheter 195 (FIG. 2B). Optionally, the measurement device 120 including the a multi-core optical fiber 135 can be formed integrally with the catheter 195. Herein, the measurement device 120 features the stylet 130, which includes an insulating layer 210 encasing a multi-core optical fiber 135 and/or a magnetic, electro-magnetic, or electrically conductive medium 230 as shown in FIGS. 6A-7 and described herein. The stylet 130 extends distally from a handle 240 while an interconnect (e.g. tether) 250 extends proximally from the handle 240 and is terminated by the console connector 132 for coupling to the interconnect 140 of the console 110 as shown in FIG. 1.

As shown, the stylet 130 and the interconnect 250 provide a pathway for outgoing optical signals produced by the light source 182 of the optical logic 180 and returning optical signals, produced by gratings within the core fibers of the multi-core optical fiber 135, for receipt by the photodetector 184 (see FIG. 1). Insulating layers associated with the stylet 130 and the interconnect 250 may vary in density and material to control its rigidity, flexibility, malleability, and other mechanical properties.

Furthermore, according to one embodiment of the disclosure, the measurement device 120 further includes a catheter connector 270, which may be threaded for attachment to a connector of an extension leg of a catheter (see FIG. 2B). This connectivity between the connector 270 and a connector of the extension leg connector may be used for coupling the measurement device 120 with the catheter 195, as shown in FIG. 2B. Note further that, it should appreciated that the term “stylet,” as used herein, can include any one of a variety of devices configured for removable placement within a lumen of the catheter 195 (or other portion of a medical device). Also, note that other connection schemes between the stylet 130 and the console 110 can also be used without limitation.

Referring to FIG. 2B, an embodiment of the stylet 130 for placement within the catheter 195 is shown. Herein, the catheter 195 includes an elongate catheter tube 300 defining one or more lumens 310 extending between proximal and distal ends of the catheter tube 300. The catheter tube 300 is in communication with a corresponding extension leg 320 via a bifurcation hub 330. Luer connectors 340 are included on the proximal ends of the extension legs 320.

As shown, the measurement device 120 includes the console connector 132 on its proximal end 350 to enable the stylet 130 to operably connect with the console 110 (see FIG. 1). The interconnect 250 distally extends communications from the console 110 to the catheter connector 270, which is configured to threadably engage (or otherwise connect with) the Luer connector 340 of one of the extension legs 320 of the catheter 195. The stylet 130 extends distally from the catheter connector 270 up to a distal-end 280 of the stylet 130. The distal-end 280 of the stylet 130 may be substantially co-terminal with a distal tip 360 of the catheter 195.

### Catheter Measuring

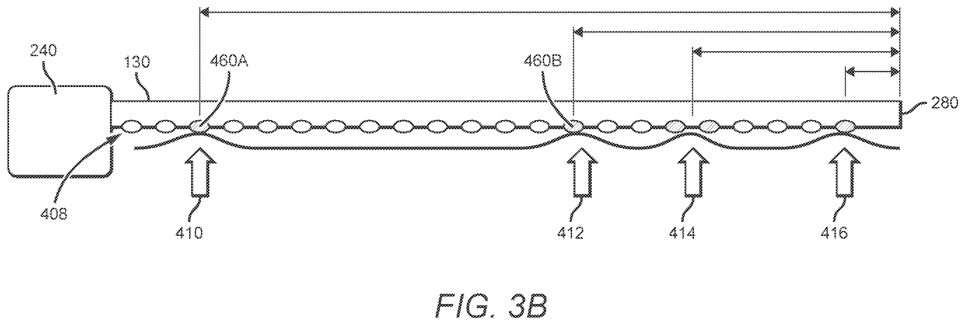

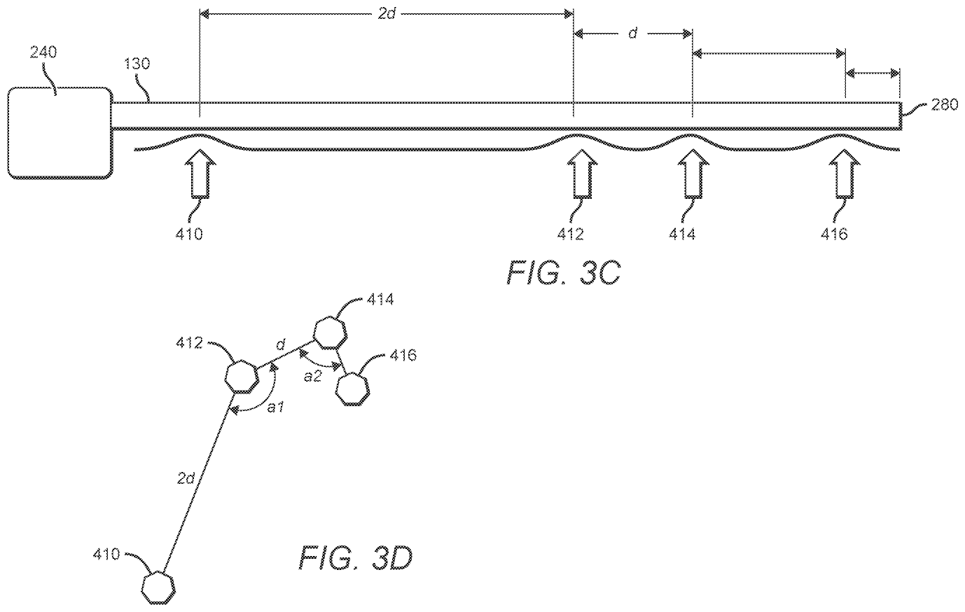

FIG. 3A shows an exemplary environment of use 10 for an automated catheter measurement system 100 as described herein. As noted, conventional methods of estimating a catheter length prior to insertion includes measuring line-of-sight distances between one or more external landmarks, such as the insertion site 410, the shoulder 412, the clavicular head 414, and the third intercostal space 416 of a patient 400. From these measurements a clinician estimates a length of the intravascular pathway between the insertion site 410 and the target location, (e.g. adjacent the third intercostal space 416). As will be appreciated, errors in estimating the intravascular distance can occur leading to misplacement of the distal tip 360 of the catheter 195.

In an embodiment, the measurement device 120 (FIGS. 2A-2B), or more specifically the stylet 130 portion of the measurement device 120 that includes the multi-core optical fiber 135 and/or magnetic medium 230 can be placed on an external surface of a patient 400 and aligned with one or more external physical landmarks, such as the insertion site 410, the shoulder 412, the clavicular head 414, and the third intercostal space 416. As will be appreciated, these are exemplary physical landmarks and not intended to be limiting. When the measuring device 120 is aligned as such, the system 100 can determine a three-dimensional arrangement of these landmarks and provide a digital framework for a predicted intravascular pathway between the insertion site 410 and a target location.

In an embodiment, the measurement device 120 can formed as a separate structure from that of the catheter 195, and can be coupled therewith to provide a measurement device 120 and catheter 195 assembly (FIG. 2B). As such, once the measurement device 120 has determined a digital framework, as described herein, the measurement device 120 can be coupled with the catheter 195 to map an actual intravascular pathway, and/or facilitate placement of the catheter 195.

In an embodiment, the measurement device 120, or more specifically the stylet 130 including the multi-core optical fiber 135 and/or magnetic medium 230 can be formed integrally with the catheter 195 and the catheter 195 and measurement device 120 assembly can be aligned with one or more external landmarks to determine a digital framework, prior to placement of the catheter 195 intravascularly.

In an embodiment, the measurement device 120 can display flexible or malleable physical characteristics to allow a user to shape the measurement device 120 into a shape and for the measurement device 120 to remain in that shape until reshaped. As such, the user can align a portion of the measurement device 120 with a first landmark, e.g. insertion site 410, and the measurement device 120 can remain in place while a second portion of the measurement device 120 is aligned with a second landmark, e.g. the shoulder 412, etc.

In an embodiment, the system 100 (e.g. the shape sensing analytic logic 192, magnetic signature tracking logic 196, etc.), can determine and record when a portion of the measurement device 120 is aligned with a physical landmark by receiving one or more inputs such as voice activation, button actuation, sensor activation, determination of bending pattern or frequency relative to a sensing region (e.g. a distal tip 280), inputs to a user interface (UI), combinations thereof or the like.

For example, a clinician can align a first portion of the measurement device 120 with a first landmark (e.g. insertion site 410) and provide one or more voice commands to the system 100 to indicate as such. The system 100 can then record the location of the first landmark in three dimensional space. The clinician can then align a second portion of the measurement device 120 with a second landmark (e.g. shoulder 412) and provide one or more voice commands to the system 100 to indicate as such. The system 100 can then record the location of the second landmark in three dimensional space, relative to the first location of the first landmark. The clinician can repeat the process until all of the positions of the landmarks are recorded. Alternatively, or in addition to, the clinician can actuate a physical actuator (button, lever, switch, etc.) or provide in input to a user interface (mouse click, touch screen, etc.) to indicate when a portion of the measurement device 120 is aligned with a physical landmark.

In an embodiment, as shown in FIG. 3B, the measurement device 120 can determine when a portion of the stylet 130 is aligned with a landmark based on an activation of a sensor 408 disposed on the device itself. For example, the stylet 130 can include an array of pressure sensors 408 extending along a longitudinal length thereof, a sensor 406 of the array of sensors 408 can activate when compressed against a physical landmark 410,412, 414, 416. Consecutive sensors of the array of sensors can be activated to indicate a relative position of the consecutive landmarks. For example a first sensor 406A can be activated to indicate a first landmark, e.g. insertion site 410, a second sensor 406B can be activated to indicate a second landmark, e.g. shoulder 412, etc. The system 100 can then determine which landmarks and the relative location based on the order of activation, and/or the proximity of the activated sensor 406 relative to either the handle 240 at a proximal end and/or a distal tip 280 of the stylet 130.

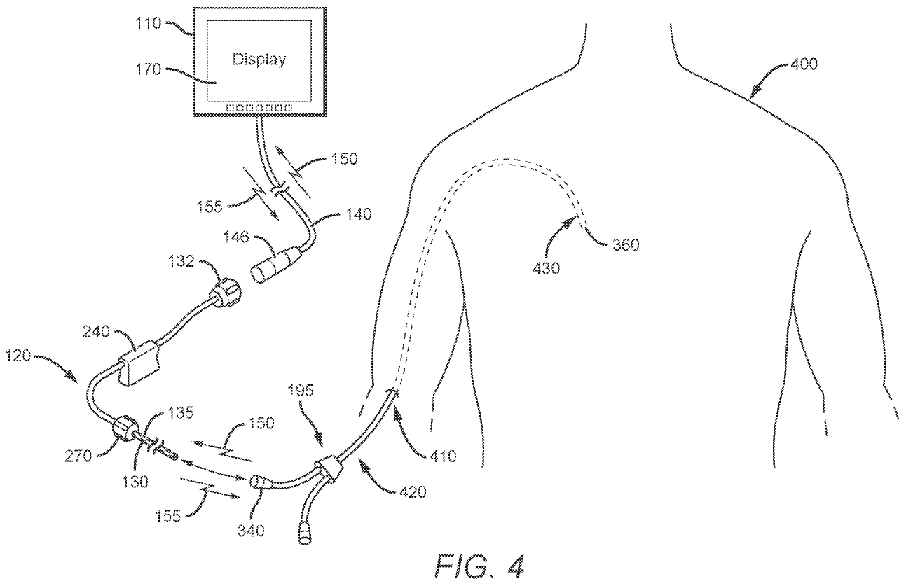

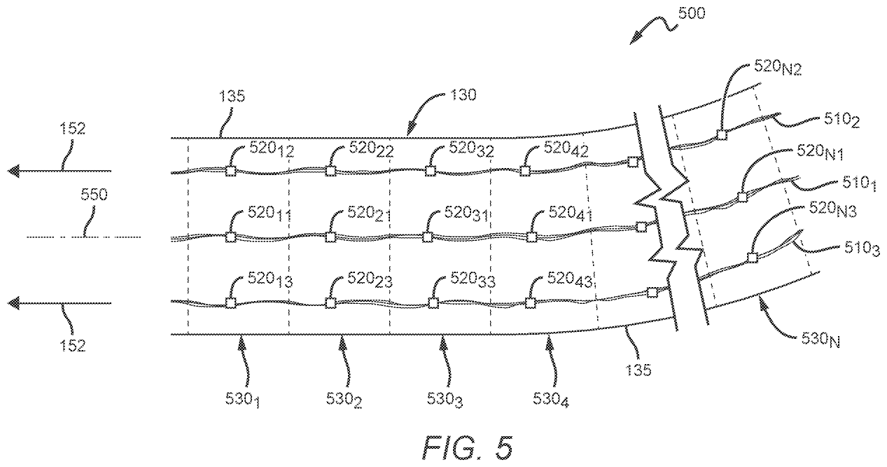

In an embodiment, as shown in FIGS. 3C-3D the system 100 can determine when a portion of the stylet 130 is aligned with a landmark based on detecting a recognized inflection pattern, frequency of inflection, relative location of an inflection, or combinations thereof, along a longitudinal length of stylet 130. To note, as described in more detail herein, the multi-core optical fiber 135 can determine a location, angle, and direction of an inflection along a length thereof (FIGS. 4-5). As used herein a location of an inflection relates to a longitudinal position of the inflection along the stylet 130. An angle of the inflection is the degree of deflection from a longitudinal axis. A direction of the inflection is a radial direction of the inflection from a central longitudinal axis.

Similarly, as shown in FIG. 3D the relative locations of the landmarks in three-dimensional space means that each landmark can be identified based on a distinct combination of distance, angle, and direction relative to each other. For example, a distance between the insertion site 410 and the shoulder 412 is proportionally larger (e.g. 2d) than a distance between the shoulder 412 and the clavicle head 414 (e.g. d). Similarly, an inflection angle (a2) at the clavicle head 414 can be different from than an inflection angle (a1) at the shoulder 412. As such, the system 100 can determine a longitudinal location of an inflection either relative to other inflections and/or relative to a proximal end 240 or distal tip 280, an order of the inflections, an angle of inflection, and/or a direction of the inflection relative to the other inflection points, and can determine which inflection point is aligned with each landmark. In an embodiment, the system 100 can be configured to automatically record the location of the landmark(s) and also be configured to receive an input from the user to update, modify, or improve the accuracy of the location of the landmark.

Once all of the landmarks have been recorded, the system 100 can determine a digital framework to map a relative location of the landmarks in three-dimensional space (FIG. 3D). The system 100 can use this framework to calculate an estimated intravascular pathway between the insertion site and the target location, and provide an initial or predicted length (L_{P}) of the catheter 195 prior to insertion of the catheter 195. Based on the predicted catheter length (L_{P}) displayed by the system 100, the user can select a specific catheter device for insertion and/or trim the catheter 195 to a suitable length. Alternatively, where the stylet 130 is formed integrally with the catheter 195, the catheter 195 can be trimmed to the predicted catheter length (L_{P}) prior to insertion. The clinician can then proceed to place the catheter 195 intravascularly.

In an embodiment, the measurement device 120 used to measure the external landmarks of the patient 400 can be included with the catheter 195 during the placement procedure to confirm the actual length (L_{A}) of the intravascular pathway compared with the predicted length of the intravascular pathway (i.e. predicted length (L_{P}) of the catheter). Advantageously, the system 100 can gather information from one or more predicted lengths (L_{P}) and one or more actual lengths (L_{A}) of the intravascular pathway and can improve the accuracy of future digital frameworks. Further, the information of predicted lengths (L_{P}) and actual lengths (L_{A}) from one or more systems 100 can be shared and analyzed over a network 12 to further increase the accuracy of the digital framework.

### Catheter Placement

Referring now to FIG. 4, an embodiment of the stylet 130 illustrating its placement within the catheter 195 as the catheter 195 is being inserted into a vasculature of a patient 400 through a skin insertion site 410 is shown. As illustrated in FIG. 4, the catheter 195 generally includes a proximal portion 420 that generally remains exterior to the patient 400 and a distal portion 430 that generally resides within the patient vasculature after placement is complete.

In an embodiment, the measurement device 120 can be employed to assist in the positioning of the distal tip 360 of the catheter 195 in a desired target location within the patient vasculature. In one embodiment, the target location for the catheter distal tip 360 is proximate the patient's heart, such as in the lower one-third (⅓^{rd}) portion of the Superior Vena Cava (“SVC”) for this embodiment. Of course, the measurement device 120 can be employed to place the catheter distal tip 360 in other locations.

During advancement of the catheter 195, the stylet 130 receives broadband light 155 from the console 110 via interconnect 140, which includes the connector 146 for coupling to the console connector 132 for the measurement device 120. The reflected light 150 from sensors (reflective gratings) within each core fiber of the multi-core optical fiber 135 are returned from the stylet 130 over the interconnect 140 for processing by the console 120. The physical state of the stylet 130 may be ascertained based on analytics of the wavelength shifts of the reflected light 150. For example, the strain caused through bending of the stylet 130, and hence angular modification of each core fiber, causes different degrees of deformation. The different degrees of deformation alters the shape of the sensors (reflective grating) positioned on the core fiber, which may cause variations (shifts) in the wavelength of the reflected light from the sensors positioned on each core fiber within the multi-core optical fiber 135, as shown in FIG. 5. From this wavelength shifting, the shape sensing analytic logic 192 within the console 110 (see FIG. 1) may determine the physical state of the stylet 130 (e.g., shape, orientation, etc.).

Referring to FIG. 5, an exemplary embodiment of a right-sided, longitudinal view of a section 500 of the multi-core optical fiber 135 included within the stylet 130 is shown. The multi-core optical fiber section 500 depicts certain core fibers 510₁-510_{M} (M≥2, M=4 as shown) along with the spatial relationship between sensors (e.g., reflective gratings) 520₁₁-520_{NM} (N≥2; M≥2) present within the core fibers 510₁-510_{M}, respectively. As shown, the section 500 is subdivided into a plurality of cross-sectional regions 530₁-530_{N}, where each cross-sectional region 530₁-530_{N} corresponds to reflective gratings 520₁₁-520₁₄ . . . 520_{N1}-520_{N4}. Some or all of the cross-sectional regions 530₁ . . . 530_{N} may be static (e.g., prescribed length) or may be dynamic (e.g., vary in size among the regions 530₁ . . . 530_{N}). A first core fiber 510₁ is positioned substantially along a center (neutral) axis 550 while core fiber 510₂ may be oriented within the cladding of the multi-core optical fiber 130, from a cross-sectional, front-facing perspective, to be position on “top” the first core fiber 510₁. In this deployment, the core fibers 510₃ and 510₄ may be positioned “bottom left” and “bottom right” of the first core fiber 510₁.

Referencing the first core fiber 510₁ as an illustrative example, when the stylet 130 is operative, each of the reflective gratings 520₁-520_{N} reflect light for a different spectral width. As shown, each of the gratings 520₁ᵢ-520_{Ni} (1≤i≤M) is associated with a different, specific spectral width, which would be represented by different center frequencies of f₁ . . . f_{N}, where neighboring spectral widths reflected by neighboring gratings are non-overlapping according to one embodiment of the disclosure.

Herein, positioned in different core fibers 510₂-510₃ but along at the same cross-sectional regions 530-530_{N} of the multi-core optical fiber 135, the gratings 520₁₂-520_{N2} and 520₁₃-520_{N3} are configured to reflect incoming light at same (or substantially similar) center frequency. As a result, the reflected light returns information that allows for a determination of the physical state of the optical fiber 135 (and the stylet 130) based on wavelength shifts measured from the returned, reflected light. In particular, strain (e.g., compression or tension) applied to the multi-core optical fiber 135 (e.g., at least core fibers 510₂-510₃) results in wavelength shifts associated with the returned, reflected light. Based on different locations, the core fibers 510₁-510₄ experience different types and degree of strain based on angular path changes as the stylet 130 advances in the patient.

For example, with respect to the multi-core optical fiber section 500 of FIG. 5, in response to angular (e.g., radial) movement of the stylet 130 is in the left-veering direction, the fourth core fiber 510₄ (see FIG. 6A) of the multi-core optical fiber 135 with the shortest radius during movement (e.g., core fiber closest to a direction of angular change) would exhibit compression (e.g., forces to shorten length). At the same time, the third core fiber 510₃ with the longest radius during movement (e.g., core fiber furthest from the direction of angular change) would exhibit tension (e.g., forces to increase length). As these forces are different and unequal, the reflected light from reflective gratings 520_{N2} and 520_{N3} associated with the core fiber 510₂ and 510₃ will exhibit different changes in wavelength. The differences in wavelength shift of the reflected light signals 152 can be used to extrapolate the physical configuration of the stylet 130 by determining the degrees of wavelength change caused by compression/tension for each of the periphery fibers (e.g., the second core fiber 510₂ and the third core fiber 510₃) in comparison to the wavelength of the reference core fiber (e.g., first core fiber 510₁) located along the neutral axis 550 of the multi-core optical fiber 135. These degrees of wavelength change may be used to extrapolate the physical state of the stylet 130.

Referring now to FIG. 6A, an exemplary embodiment of the multimodal stylet 130 of FIG. 1 supporting both an optical and electrical/magnetic signaling is shown. Herein, the stylet 130 features a centrally located multi-core optical fiber 135, which includes a cladding 600 and a plurality of core fibers 510₁-510_{M} (M≥2; M=4) residing within a corresponding plurality of lumens 620₁-620_{M}. While the multi-core optical fiber 135 is illustrated within four (4) core fibers 510₁-510₄, a greater number of core fibers 510₁-510_{M} (M≥4) may be deployed to provide a more detailed three-dimensional sensing of the physical state (e.g., shape, orientation, etc.) of the multi-core optical fiber 135 and the stylet 130 deploying the optical fiber 135, a greater number of core fibers 510₁-510_{M} (M≥4) may be deployed.

For this embodiment of the disclosure, the multi-core optical fiber 135 is encapsulated within a concentric braided tubing 610 positioned over a low-coefficient of friction layer 635. The braided tubing 610 may feature a “mesh” construction, in which the spacing between the intersecting conductive elements is selected based on the degree of rigidity/flexibility, or elasticity/malleability desired for the stylet 130. For example, a greater spacing may provide a lesser rigidity, and thereby, a more pliable stylet 130.

According to this embodiment of the disclosure, as shown in FIGS. 6A-6B, the core fibers 510₁-510₄ include (i) a central core fiber 510₁ and (ii) a plurality of periphery core fibers 510₂-510₄, which are maintained within lumens 620₁-620₄ formed in the cladding 600. According to one embodiment of the disclosure, one or more of the lumen 620₁-620₄ may be configured with a diameter sized to be greater than the diameter of the core fibers 510₁-510₄. By avoiding a majority of the surface area of the core fibers 510₁-510₄ from being in direct physical contact with a wall surface of the lumens 620₁-620₄, the wavelength changes to the incident light are caused by angular deviations in the multi-core optical fiber 135 thereby reducing influence of compression and tension forces being applied to the walls of the lumens 620₁-620_{M}, not the core fibers 510₁-510_{M} themselves.

As further shown in FIGS. 6A-6B, the core fibers 510₁-510₄ may include central core fiber 510₁ residing within a central or first lumen 620₁ formed along the first neutral axis 550 and a plurality of core fibers 510₂-510₄ residing within lumens 620₂-620₄ each formed within different areas of the cladding 600 radiating from the first neutral axis 550. In general, the core fibers 510₂-510₄, exclusive of the central core fiber 510₁, may be positioned at different areas within a cross-sectional area 605 of the cladding 600 to provide sufficient separation to enable three-dimensional sensing of the multi-core optical fiber 135 based on changes in wavelength of incident light propagating through the core fibers 510₂-510₄ and reflected back to the console for analysis.

For example, where the cladding 600 features a circular cross-sectional area 605 as shown in FIG. 6B, the core fibers 510₂-510₄ may be positioned substantially equidistant from each other as measured along a perimeter of the cladding 600, such as at “top” (12 o'clock), “bottom-left” (8 o'clock) and “bottom-right” (4 o'clock) locations as shown. Hence, in general terms, the core fibers 510₂-510₄ may be positioned within different segments of the cross-sectional area 605. Where the cross-sectional area 605 of the cladding 600 features a polygon cross-sectional shape (e.g., triangular, square, rectangular, pentagon, hexagon, octagon, etc.), the central core fiber 510₁ may be located at or near a center of the polygon shape, while the remaining core fibers 510₂-510_{M} may be located proximate to angles between intersecting sides of the polygon shape.

Referring still to FIGS. 6A-6B, operating as the conductive medium for the stylet 130, the braided tubing 610 provides mechanical integrity to the multi-core optical fiber 135 and operates as a conductive pathway for magnetic, electro-magnetic, or electrical signals. For example, the braided tubing 610 may be exposed to a distal tip 630 of the stylet 130. The cladding 600 and the braided tubing 610, which is positioned concentrically surrounding a circumference of the cladding 600, are contained within the same insulating layer 650. The insulating layer 650 may be a sheath or conduit made of protective, insulating (e.g., non-conductive) material that encapsulates both for the cladding 600 and the braided tubing 610, as shown.

Further examples and embodiments of fiber-optical enable shape sensing devices can be found in, U.S. 2018/0289927, U.S. 2021/0045814, U.S. 2021/0156676, U.S. 2021/0154440, U.S. 2021/0275257, U.S. 2021/0298680, U.S. 2021/0268229, U.S. 2021/0271035, U.S. 2021/0402144, U.S. 2021/0401509, U.S. 2022/0011192, U.S. 2022/0034733, U.S. 2022/0110695, U.S. 2022/0160209, U.S. 2022/0152349, U.S. 2022/0110706, U.S. 2022/0211442, and U.S. 2022/0233246, each of which are incorporated in their entirety herein.

### Magnetic Signature Tracking

FIG. 7 shows further details of a magnetic signature enabled measurement device 120. In an embodiment, the stylet 130 can include one or more regions of magnetic medium 230 disposed along a longitudinal length of the stylet 130. For example a first magnetic signature region 706A, a second magnetic signature region 706B, a third magnetic signature region 706C, and a fourth magnetic signature region 706D. Each individual region 706A, 706B, 706C, 706D can include a unique dipole pattern of magnetic material. For example, as shown in FIG. 7 a white region can be a South to North pole orientation and a black region can be a North to South pole orientation. However, as will be appreciated this is exemplary and not intended to be limiting. In an embodiment, each magnetic signature region can be predetermined. Alternatively, the magnetic medium 230 can be exposed to a magnetic field to induce a distinct dipole pattern to each magnetic signature region.

The system 100, including the magnetic signature logic 196, can detect and identify each of these magnetic signature regions 706A, 706B, 706C, 706D based on the unique dipole pattern of magnetic material. The system 100 can then triangulate and track each separate region simultaneously in three-dimensional space. In an embodiment, the one or more magnetic regions 706 can be distributed evenly along the length of the stylet 130. The system 100 can then track a physical state of the stylet 130 when the stylet 130 is aligned with the one or more external landmarks, as described herein. In an embodiment, as shown in FIG. 7, each magnetic region 706 can be aligned with a predetermined longitudinal position on the stylet 130. Each predetermined longitudinal position can then be aligned with an external landmark to provide a three-dimensional map, as described herein. In an embodiment, each predetermined longitudinal position can include an alphanumeric symbol and/or color coded to indicate to a user which predetermined longitudinal position of the stylet 130 should be aligned with which each external landmark.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A system for determining a catheter length required to extend between an insertion site and a target location prior to placement within a patient body, the system comprising:
a measurement device comprising an optical fiber having one or more of core fibers and configured to be aligned with one or more external landmarks; and
a console including one or more processors and a non-transitory computer-readable medium having stored thereon logic, when executed by the one or more processors, causes operations including:
providing an incident light signal to the optical fiber;
receiving a reflected light signal of the incident light;
processing the reflected light signal to determine a location of one or more external landmarks in three-dimensional space; and
determining a predicted catheter length required to extend intravascularly between the insertion site and the target location.

2. The system according to claim 1, wherein the measurement device includes a stylet having a multi-core optical fiber extending therethrough and one or both of a magnetic element and an electrically conductive element extending therethrough.

3. The system according to claim 1, wherein an external landmark of the one or more external landmarks includes an insertion site, a shoulder, a clavicle head, and a third intercostal space.

4. The system according to claim 1, wherein the console is further configured to receive an input to indicate when a portion of the measurement device is aligned an external landmark of the one or more external landmarks.

5. The system according to claim 1, wherein the measurement device further includes a pressure sensor array extending along a longitudinal length thereof, and wherein activation of a sensor of the pressure sensor array indicates that a portion of the measurement device is aligned with an external landmark of the one or more external landmarks.

6. The system according to claim 1, wherein the console is further configured to process the reflected light signal to determine a location, angle, and direction of one or more inflections in the measurement device and determine a location of an external landmark of the one or more external landmarks in three-dimensional space.

7. The system according to claim 1, wherein the measurement device displays malleable physical qualities and can be shaped into a shape and remain in that shape until reshaped.

8. The system according to claim 1, wherein determining the predicted catheter length further includes determining one or more of a distance, an angle, and a direction between a first external landmark and a second external landmark.

9. A method of determining a catheter length required to extend between an insertion site and an intravascular target location prior to placement within a patient body, comprising:
aligning a measurement device with a first external landmark and a second external landmark on a surface of the patient body, the measurement device including one or both of an optical fiber having one or more of core fibers and a magnetic signature region;
mapping a three dimensional location of the first external landmark relative to the second external landmark; and
determining a predicted length (L_{P}) of an intravascular pathway between the insertion site and the intravascular target location that extends proximate the first landmark and the second landmark.

10. The method according to claim 9, further including extending the measurement device intravascularly from the insertion site to the target location and mapping an intravascular pathway therebetween to determine an actual length (L_{A}) of the intravascular pathway between the insertion site and the target location.

11. The method according to claim 9, wherein the measurement device is configured to be placed within a lumen of a catheter.

12. The method according to claim 9, wherein the measurement device includes a stylet having a multi-core optical fiber extending therethrough and one or both of a magnetic element and an electrically conductive element extending therethrough.

13. The method according to claim 9, wherein the first external landmark or the second external landmark includes one of an insertion site, a shoulder, a clavicle head, and a third intercostal space.

14. The method according to claim 9, wherein aligning a measurement device further includes receiving an input to indicate when a portion of the measurement device is aligned one of the first external landmark or the second external landmark.

15. The method according to claim 9, wherein the measurement device further includes a pressure sensor array extending along a longitudinal length thereof, and wherein the method further includes activation of a sensor of the pressure sensor array to indicate that a portion of the measurement device is aligned with one of the first external landmark or the second external landmark.

16. The method according to claim 9, wherein mapping a three dimensional location of a first external landmark or a second external landmark further includes processing a reflected light signal to determine a location, angle, and direction of an inflection in the measurement device.

17. The method according to claim 9, wherein aligning a measurement device further includes shaping the measurement device into a shape, the measurement device remaining in that shape until reshaped.

18. The method according to claim 9, wherein mapping a three dimensional location of a first external landmark or a second external landmark further includes identifying a first magnetic signature region and a second magnetic signature region and tracking the first magnetic signature region and a second magnetic signature region in three-dimensional space.
